(19) United States
(12) Patent Application Publication
(10) Pub. No.: US 2026/0276627 A1
(43) Pub. Date: Sep. 17, 2026

(54) METHODS FOR INHIBITING MACROPHAGE NEUROTOXICITY

(71) Applicant: AJOU UNIVERSITY INDUSTRY-ACADEMIC COOPERATION FOUNDATION, Suwon-si (KR)

(72) Inventors: Byung Gon KIM, Seoul (KR); Yeo Jin SEO, Changwon-si (KR); Hyung Soon KIM, Gwangju-si (KR); Seung Ah JEE, Gumi-si (KR)

(21) Appl. No.: 19/660,285

(22) Filed: Apr. 28, 2026

Related U.S. Application Data
(63) Continuation of application No. PCT/KR2024/017233, filed on Nov. 5, 2024.

(30) Foreign Application Priority Data
Nov. 10, 2023 (KR) 10-2023-0155384
Nov. 4, 2024 (KR) 10-2024-0154221

(51) Int. Cl. G01N 33/50; C12N 5/0786; C12N 9/12; C12Q 1/48

(52) U.S. Cl. CPC G01N 33/5023; C12N 5/0645; C12N 9/12; C12Q 1/485; G01N 33/5055; C12N 2502/081; C12Y 207/11001; G01N 2333/912; G01N 2500/10; G01N 2800/2871

(57) ABSTRACT

Provided is a method for inhibiting macrophage neurotoxicity, the method comprising increasing expression of Aurora kinase in macrophages, wherein the Aurora kinase includes AURKA, AURKB, or a combination thereof. The expression may be increased by co-culturing the macrophages with neurons such as dorsal root ganglion sensory neurons, hippocampal neurons, dopaminergic neurons, motor neurons, cerebellar granule cells, thalamic neurons, or spinal cord neurons.

### FIELD

The present disclosure relates to a method for inhibiting macrophage neurotoxicity.

### DESCRIPTION OF THE RELATED ART

The central nervous system of vertebrates is protected by the skull and spine, but once damaged, its recovery is very limited. Central nervous system damage, such as stroke, occurs due to impaired blood supply caused by thrombi or the like, which results in severe damage to neural tissue. To date, groundbreaking therapies capable of effectively inhibiting damage to neural tissue and promoting recovery of neurological function at an early stage of stroke onset are very limited.

Damage caused by stroke is further expanded by secondary degeneration that progresses over several days after primary injury. In particular, in the case of ischemic stroke, the penumbra region around the infarct core is an important region that may be recoverable through appropriate treatment. According to recent studies, inflammatory responses occurring after stroke, particularly activation of macrophages, have attracted attention as a major cause of secondary injury.

Macrophages are important components of the immune system and, under normal conditions, serve to remove damaged tissue and promote healing. However, in acute injury conditions such as stroke, excessively activated macrophages may rather secrete neurotoxic substances, thereby causing additional neuronal damage. Such macrophage neurotoxicity aggravates inflammatory responses after stroke and consequently acts as a major factor that inhibits recovery of neural tissue.

Accordingly, there is an urgent need to develop a new therapeutic method capable of effectively inhibiting macrophage neurotoxicity after stroke to regulate inflammatory responses, thereby minimizing damage to neural tissue and promoting long-term recovery of neurological function.

Korean Patent Publication No. 10-2021-0151945, which is a background art of the present disclosure, relates to a method for modulating macrophage activity.

### DESCRIPTION

### Technical Problem

The present disclosure is directed to solving the problems of the prior art described above and provides a method for inhibiting macrophage neurotoxicity.

The present disclosure also provides a pharmaceutical composition for inhibiting neural tissue degeneration.

The present disclosure also provides a biomarker for diagnosing a neurodegenerative disease.

The present disclosure also provides a kit for diagnosing a neurodegenerative disease, comprising the biomarker for diagnosing the neurodegenerative disease.

The present disclosure also provides a method for screening an inhibitor of macrophage neurotoxicity.

However, the technical problems to be achieved by the embodiments of the present disclosure are not limited to the technical problems described above, and there may be other technical problems.

### Means for Solving the Problem

As a technical means for achieving the aforementioned technical problems, a first aspect of the present disclosure provides a method for inhibiting macrophage neurotoxicity, comprising increasing expression of Aurora kinase in macrophages.

According to one embodiment of the present disclosure, the Aurora kinase may comprise one selected from the group consisting of AURKA, AURKB, and combinations thereof, but is not limited thereto.

According to one embodiment of the present disclosure, the increasing of the expression may comprise co-culturing the macrophages with neurons, but is not limited thereto.

According to one embodiment of the present disclosure, the neurons may comprise one selected from the group consisting of dorsal root ganglion sensory neurons, hippocampal neurons, dopaminergic neurons, motor neurons, cerebellar granule cells, thalamic neurons, spinal cord neurons, and combinations thereof, but are not limited thereto.

In addition, a second aspect of the present disclosure provides a pharmaceutical composition for inhibiting neural tissue degeneration, comprising an active substance that increases expression of Aurora kinase.

According to one embodiment of the present disclosure, the Aurora kinase may comprise one selected from the group consisting of AURKA, AURKB, and combinations thereof, but is not limited thereto.

In addition, a third aspect of the present disclosure provides a biomarker for diagnosing a neurodegenerative disease, comprising Aurora kinase as an active ingredient.

According to one embodiment of the present disclosure, the Aurora kinase may comprise one selected from the group consisting of AURKA, AURKB, and combinations thereof, but is not limited thereto.

According to one embodiment of the present disclosure, when downregulation of an expression level of the Aurora kinase is observed, a subject may be determined to have a neurodegenerative disease, but is not limited thereto.

According to one embodiment of the present disclosure, the neurodegenerative disease may comprise one selected from the group consisting of stroke, Alzheimer's disease, Parkinson's disease, Huntington's disease, and combinations thereof, but is not limited thereto.

According to one embodiment of the present disclosure, the neurodegenerative disease may comprise stroke, but is not limited thereto.

In addition, a fourth aspect of the present disclosure provides a kit for diagnosing a neurodegenerative disease, comprising the biomarker for diagnosing a neurodegenerative disease according to the third aspect of the present disclosure.

In addition, a fifth aspect of the present disclosure provides a method for screening an inhibitor of macrophage neurotoxicity, comprising: treating macrophages with a candidate substance; measuring an expression level of Aurora kinase in the macrophages; and selecting the candidate substance as an inhibitor of macrophage neurotoxicity when expression of the Aurora kinase is increased.

According to one embodiment of the present disclosure, the Aurora kinase may comprise one selected from the group consisting of AURKA, AURKB, and combinations thereof, but is not limited thereto.

The aforementioned means for solving the problem are merely exemplary and should not be construed as intending to limit the present disclosure. In addition to the exemplary embodiments described above, further embodiments may exist in the drawings and the detailed description of the invention.

### Effect of the Invention

The present invention presents new possibilities in the field of research on inhibition of secondary degeneration caused by macrophage inflammatory responses and development of therapeutic drugs, which are currently insufficient, and experimentally demonstrated that neurotoxicity can be regulated and neuronal damage can be reduced by regulating expression of Aurora kinase in infiltrating macrophages after ischemic stroke. Accordingly, the present invention can provide a new approach capable of inducing inhibition of secondary degeneration after central nervous system damage.

In addition, the present invention has important significance in clinical settings after stroke, and in a situation where prognostic tools for monitoring current neurological status and predicting final outcomes are lacking, the present research results can provide a basis for developing new prognostic tools. Furthermore, in the current situation where there are no stroke-related blood biomarkers available for clinical use, the present research can suggest the possibility of developing new biomarkers capable of monitoring inflammatory responses.

In addition, the present invention can inhibit neurotoxicity in other forms of neural damage and neurodegenerative diseases in addition to ischemic damage caused by inflammatory responses of macrophages, thereby presenting a therapeutic platform that can be commonly used for various neurological diseases.

However, the effects obtainable from the present disclosure are not limited to the effects described above, and other effects may also be present.

### BRIEF DESCRIPTION OF THE DRAWING

FIG. 1 shows results confirming neuronal cell death caused by neurotoxicity when a macrophage culture medium according to one experimental example of the present disclosure is treated to neurons.

FIG. 2 shows results confirming reduced neurotoxicity when a culture medium of macrophages co-cultured with neurons according to one experimental example of the present disclosure is treated to neurons.

FIG. 3A shows results confirming an increase in cell cycle-related mechanisms by analyzing genomes increased in co-cultured macrophages using IPA (Ingenuity Pathway Analysis).

FIG. 3B shows results confirming, by examining a network of genomes increased in co-cultured macrophages using Cytoscape, that cell cycle-related genes are included in core proteins of the network.

FIG. 3C shows results confirming an increase in cell cycle-related mechanisms by analyzing genomes increased in co-cultured macrophages using a GSEA database.

FIG. 3D shows results confirming that many genes are associated with cell cycle mechanisms in GSEA database analysis.

FIG. 3E shows results confirming, through PCR, that cell cycle genes are increased in co-cultured macrophages in transcriptome analysis.

FIG. 4A shows results of observing expression of AURKA, AURKB, and AURKC in transcriptome results.

FIG. 4B shows results of observing, through PCR, that expression of AURKA and AURKB is increased in co-cultured macrophages.

FIG. 4C shows the quantified real-time PCR results corresponding to FIG. 4b, confirming increased expression of AURKA and AURKB in co-cultured macrophages.

FIG. 4D shows results of observing, through Western blot, that expression of AURKA and AURKB is increased in co-cultured macrophages.

FIG. 4E shows results obtained by quantifying the Western blot results of FIG. 4D using ImageJ, thereby confirming that expression of AURKA and AURKB is increased in co-cultured macrophages.

FIG. 4F shows results of observing, through PCR, that expression of AURKA and AURKB, which had been increased in co-cultured macrophages, is decreased upon treatment with a cell cycle inhibitor.

FIG. 4G shows the quantified real-time PCR results corresponding to FIG. 4f, confirming increased expression of AURKA and AURKB in co-cultured macrophages.

FIG. 4H shows results of observing, through Western blot, that expression of AURKA and AURKB, which had been increased in co-cultured macrophages, is decreased upon treatment with a cell cycle inhibitor.

FIG. 4I shows results obtained by quantifying the Western blot results of FIG. 4H using ImageJ.

FIG. 5A shows results confirming that neurotoxicity is increased when expression of AURKA or AURKB is inhibited in macrophages using specific inhibitor, a culture medium is obtained, and the culture medium is treated to neurons.

FIG. 5B shows results confirming that neurotoxicity is decreased when a plasmid including an AURKA or AURKB sequence is introduced into macrophages not co-cultured with neurons (macrophages cultured alone) to overexpress AURKA or AURKB, respectively, a culture medium is obtained, and the culture medium is treated to neurons.

FIG. 5C shows results obtained by quantifying the image obtained in FIG. 5A and then calculating a D.I. (Degeneration Index).

FIG. 5D shows results obtained by quantifying the image obtained in FIG. 5B and then calculating a D.I. (Degeneration Index).

FIG. 6A is a schematic illustration of a process of inducing photothrombotic stroke in mice, and includes the meaning that damage can be observed 7 days after inducing stroke in mice.

FIG. 6B shows results confirming that damage occurred, as indicated by a circle, as a result of observing the brains of mice after 7 days.

FIG. 6C shows results confirming that a lesion occurred through CV (Cresyl Violet) staining after sectioning tissue.

FIG. 6D shows results of observing decreased expression of AURKA and AURKB in an injured region by performing Western blot using tissues obtained from Sham, Stroke Contra, and Stroke ipsi regions 7 days after injury.

### DETAILED DESCRIPTION

Hereinafter, examples of the present disclosure will be described in detail so as to be easily implemented by those skilled in the art, with reference to the accompanying drawings. However, the present disclosure may be embodied in many different forms and is not limited to the examples to be described herein. In addition, parts not related to the description have been omitted in order to clearly describe the present disclosure in the drawings, and throughout the present specification, like reference numerals designate like elements.

Throughout the present specification, when a certain part is “connected” with another part, it is meant that the certain part may be “directly connected” with the other part and may be “electrically connected” with the other part with another element interposed therebetween.

Throughout the present specification, it will be understood that when a certain member is located “on,” “above,” “at the top of,” “under,” “below,” or “at the bottom of” another member, the certain member may be in contact with the other member, and another member may also be present between the two members.

Throughout the present specification, a case where a part “includes” an element will be understood to imply the inclusion of the stated element but not the exclusion of any other elements unless explicitly described to the contrary.

The terms “about,” “substantially,” and the like used in the specification are used as a numerical value or a value close to the numerical value when inherent manufacturing and material tolerances are presented in the stated meaning, and are used to prevent an unscrupulous infringer from unfairly using disclosed contents in which precise or absolute numerical values are mentioned to help in understanding of the present disclosure. In addition, throughout the present specification, the term “step to” or “step of” does not mean “step for.”

Throughout the present specification, the term “combinations thereof” included in an expression of the Markush form means one or more mixtures or combinations selected from the group consisting of components described in the expression of the Markush form, and means to include at least one selected from the group consisting of the components.

Throughout the present specification, “A and/or B” means “A or B, or A and B.”

Hereinafter, the method for inhibiting macrophage neurotoxicity of the present disclosure will be described in detail with reference to embodiments, examples, and drawings. However, the present disclosure is not limited to these embodiments, examples, and drawings.

As a technical means for achieving the aforementioned technical problems, a first aspect of the present disclosure provides a method for inhibiting macrophage neurotoxicity, comprising increasing expression of Aurora kinase in macrophages.

The present invention presents new possibilities in the field of research on inhibition of secondary degeneration caused by macrophage inflammatory responses and development of therapeutic drugs, which are currently insufficient, and experimentally demonstrated that neurotoxicity can be regulated and neuronal damage can be reduced by regulating expression of Aurora kinase in infiltrating macrophages after ischemic stroke. Accordingly, the present invention can provide a new approach capable of inducing inhibition of secondary degeneration after central nervous system damage.

In addition, the present invention has important significance in clinical settings after stroke, and in a situation where prognostic tools for monitoring current neurological status and predicting final outcomes are lacking, the present research results can provide a basis for developing new prognostic tools. Furthermore, in the current situation where there are no stroke-related blood biomarkers available for clinical use, the present research can suggest the possibility of developing new biomarkers capable of monitoring inflammatory responses.

According to one embodiment of the present disclosure, the Aurora kinase may comprise one selected from the group consisting of AURKA, AURKB, and combinations thereof, but is not limited thereto.

According to one embodiment of the present disclosure, the increasing of the expression may comprise co-culturing the macrophages with neurons, but is not limited thereto.

According to one embodiment of the present disclosure, the neurons may comprise one selected from the group consisting of dorsal root ganglion sensory neurons, hippocampal neurons, dopaminergic neurons, motor neurons, cerebellar granule cells, thalamic neurons, spinal cord neurons, and combinations thereof, but are not limited thereto.

In addition, a second aspect of the present disclosure provides a pharmaceutical composition for inhibiting neural tissue degeneration, comprising an active substance that increases expression of Aurora kinase.

With respect to the pharmaceutical composition for inhibiting neural tissue degeneration according to the second aspect of the present disclosure, detailed descriptions of parts overlapping with the first aspect of the present disclosure have been omitted. However, even if the descriptions are omitted, the contents described in the first aspect of the present disclosure may be equally applied to the second aspect of the present disclosure.

According to one embodiment of the present disclosure, the Aurora kinase may comprise one selected from the group consisting of AURKA, AURKB, and combinations thereof, but is not limited thereto.

In addition, a third aspect of the present disclosure provides a biomarker for diagnosing a neurodegenerative disease, comprising Aurora kinase as an active ingredient.

With respect to the biomarker for diagnosing a neurodegenerative disease according to the third aspect of the present disclosure, detailed descriptions of parts overlapping with the first aspect of the present disclosure have been omitted. However, even if the descriptions are omitted, the contents described in the first aspect of the present disclosure may be equally applied to the third aspect of the present disclosure.

According to one embodiment of the present disclosure, the Aurora kinase may comprise one selected from the group consisting of AURKA, AURKB, and combinations thereof, but is not limited thereto.

According to one embodiment of the present disclosure, when downregulation of an expression level of the Aurora kinase is observed, a subject may be determined to have a neurodegenerative disease, but is not limited thereto.

According to one embodiment of the present disclosure, the neurodegenerative disease may comprise one selected from the group consisting of stroke, Alzheimer's disease, Parkinson's disease, Huntington's disease, and combinations thereof, but is not limited thereto.

According to one embodiment of the present disclosure, the neurodegenerative disease may comprise stroke, but is not limited thereto.

In addition, a fourth aspect of the present disclosure provides a kit for diagnosing a neurodegenerative disease, comprising the biomarker for diagnosing a neurodegenerative disease according to the third aspect of the present disclosure.

With respect to the kit for diagnosing a neurodegenerative disease according to the fourth aspect of the present disclosure, detailed descriptions of parts overlapping with the third aspect of the present disclosure have been omitted. However, even if the descriptions are omitted, the contents described in the third aspect of the present disclosure may be equally applied to the fourth aspect of the present disclosure.

In addition, a fifth aspect of the present disclosure provides a method for screening an inhibitor of macrophage neurotoxicity, comprising: treating macrophages with a candidate substance; measuring an expression level of Aurora kinase in the macrophages; and selecting the candidate substance as an inhibitor of macrophage neurotoxicity when expression of the Aurora kinase is increased.

With respect to the method for screening an inhibitor of macrophage neurotoxicity according to the fifth aspect of the present disclosure, detailed descriptions of parts overlapping with the first aspect of the present disclosure have been omitted. However, even if the descriptions are omitted, the contents described in the first aspect of the present disclosure may be equally applied to the fifth aspect of the present disclosure.

According to one embodiment of the present disclosure, the Aurora kinase may comprise one selected from the group consisting of AURKA, AURKB, and combinations thereof, but is not limited thereto.

Hereinafter, the present invention will be described in more detail through examples. However, the following examples are provided only for illustrative purposes and are not intended to limit the scope of the present disclosure.

### EXPERIMENTAL METHODS

### Experimental Animals

Animals used in all experiments were C57BL/6 wild-type mice purchased from Koatech Co. For cortical neuron culture, E17 pregnant mice were purchased from Koatech Co. All animal experimental procedures were conducted after approval by the Institutional Animal Care and Use Committee of Ajou University School of Medicine.

### Macrophage (BMDM, Bone Marrow-Derived Macrophage) Culture

Experiments were conducted after euthanizing mice by CO2 gas inhalation. After incising the skin covering both hind limbs, bones with attached muscles were removed and placed in a sterile Petri dish (100 mm2) containing cold PBS. After removing the muscles, the region between the femur and knee joint was cut, and bone marrow was extracted from the bone using 10 ml of PBS. The bone marrow was centrifuged at 1,200 rpm for 5 minutes, the supernatant was discarded, and the precipitated cells were diluted in DMEM medium containing 10% FBS and 1% penicillin and then cultured in a 150 mm2 Petri dish.

To increase AURKA or AURKB expression, macrophages were transfected with plasmids encoding AURKA or AURKB using electroporation. Briefly, macrophages and plasmids were mixed and subjected to electroporation, followed by incubation in culture medium. Four hours after treatment, the medium was replaced with fresh medium, and the macrophages were left for 72 hours to obtain a culture medium.

### Dorsal Root Ganglion Sensory Neuron (DRG, Dorsal Root Ganglion) Culture

Experiments were conducted after euthanizing mice by CO2 gas inhalation. After excising L3, L4, and L5 ganglia, the ganglia were dissociated into single cells with type XI collagenase (Sigma) at 37° C. for 90 minutes and then centrifuged at 1,200 rpm for 5 minutes. After centrifugation, the precipitated cells were resuspended in a culture medium supplemented with 2% B27 and then cultured in a 24-well plate for 4 hours.

For co-culture of dorsal root ganglion sensory neurons and macrophages, 4 hours after neuron culture, macrophages were cultured in a trans-well at a ratio of 1:5. After culturing for 72 hours, a culture medium was obtained.

### Primary Cortical Neuron (CN, Cortical Neuron) Culture

Neuron culture was performed using pregnant female mice at embryonic day 17. Embryos were separated from the placenta, fetal heads were cut, and the heads were transferred to a 60 mm Petri dish. Brains were isolated, meninges were removed, and cerebral cortices were dissected. The collected cerebral cortices were transferred to a 15 ml tube filled with papain enzyme (Worthington, final concentration of 14 U/ml) diluted in HBSS (Hank's Balanced Salt Solution), and digestion was performed at 37° C. for 15 minutes. After 15 minutes, the cortices were washed three times with 5 ml of HBSS solution, 1 ml of medium (CNB: Neurobasal medium, 2% B27, 1% sodium pyruvate, 1% GlutaMAX, 1% antibiotic, and 0.5% fetal bovine serum) was added, and the cortices were dissociated into single cells by pipetting 40 times. Thereafter, the cells were placed in a 24-well plate and cultured. After 1 day of culture, the medium was replaced with fresh CNB medium. To observe neurotoxicity, on day 7 of culture, the CNB medium was replaced with a macrophage culture medium. After 1 day, the culture medium was removed, the cells were fixed with 4% PFA, and immunostaining was performed.

For quantification of neurotoxicity, images were obtained at 200× magnification. The images were analyzed using ImageJ software. The stained portions in the images were quantified in their entirety, and for damaged neurons, particle areas (pixels, 10 to 10000) were set, and brightness of the corresponding areas was measured. Thereafter, a DI (Degeneration Index) was calculated by dividing the particle area by the brightness of the total stained area.

### RNA Isolation and PCR

Total RNA was isolated from cultured macrophages using TRIzol (Gibco) according to the manufacturer's protocol. Reverse transcription was performed using AccuPower PCR Premix (Bioneer). Quantitative real-time PCR was performed using SYBR Premix Ex Taq (Takara) on an Applied Biosystems 7500 Real-Time PCR system, and relative expression with respect to 18S rRNA was quantified using the comparative Ct method.

### Protein Isolation and Western Blot

Isolated tissues and macrophages were lysed by adding lysis buffer. Then, tissue extracts of the lysed tissues were centrifuged at 13,000 rpm for 15 minutes at 4° C., and the supernatant was used to measure protein concentration. Protein samples were separated on a 4-20% gel and then transferred to a PVDF membrane. Proteins transferred to the membrane were incubated with AURKA (Abcam, ab13824, 1:1000) and AURKB (Abcam, ab2254, 1:1000) antibodies, and then incubated by binding appropriate HRP-conjugated secondary antibodies. Immunoreactive bands were detected using a chemiluminescent substrate (ECL solution).

### Photothrombotic Ischemic Stroke Model (Photothrombosis Model)

A photothrombotic ischemic stroke model was produced using adult mice anesthetized with ketamine. The anesthetized animals were fixed in a stereotaxic frame, and two skin incisions were made. After incision, Rose Bengal (1010 μg/ml, Sigma, St. Louis, USA) was administered at a concentration of 100 μl/g, followed by waiting for 5 minutes for absorption into the blood stream. After 5 minutes, a light source was irradiated onto the skull for 15 minutes. KL 1500 HAL (laser) was used as the light source. The light source was irradiated onto the skull at a lateral position 1.8 mm to the left from the bregma.

### Tissue Immunostaining and Image Acquisition

Tissue samples for tissue immunostaining were prepared by anesthetizing experimental animals with ketamine and then fixing the animals through perfusion with PBS and 4% PFA. After perfusion, tissues were collected and placed in a 30% sucrose solution at 4° C. for one day. After sucrose treatment, the tissues were sectioned to a thickness of 30 μm using a frozen microtome. The sectioned tissues were attached to tissue slides, and tissue immunostaining was performed. After attachment to slides, the tissues were dried on a slide warmer for 1 hour. The dried tissue slides were immersed in dH 20 for 2 minutes and then dehydrated in alcohol concentrations of 70% (2 minutes), 90% (2 minutes), 100% (4 minutes), 90% (2 minutes), and 70% (2 minutes). After dehydration, the slides were immersed in dH 20 for 30 seconds and then stained by immersion in a Cresyl Violet solution for 2 minutes. After staining, the slides were rinsed 3 to 4 times with glacial acetic acid and then dehydrated 10 times each in alcohol concentrations of 95%, 100%, and 100%. The dehydrated and stained tissues were covered by dropping one drop of Permount on one end and pressing a coverslip thereon, dried at room temperature for one day, and then imaged. The stained tissue slides were imaged using a Zeiss LSM880 (Jena, Germany) microscope. Each tissue slide was imaged with a 100× lens at a magnification of 0.5.

### Experimental Example 1

After hematopoietic cells were extracted from mouse bone marrow, the cells were treated with M-CSF (macrophage colony stimulating factor) for 10 days and cultured to differentiate into macrophages. After one day, the medium was replaced with a fresh medium, and the cells were cultured for 72 hours. After 72 hours, a macrophage culture medium was obtained, treated to primary cultured cortical neurons, and after 48 hours, images of neurons were obtained through staining with a Tuj-1 antibody that labels neurons. In the obtained images, the degree of neurotoxicity was quantified as a D.I. (Degeneration Index), and neurotoxicity of neurons caused by the macrophage culture medium was observed. For comparison, a condition in which the culture medium was not treated was used as a control.

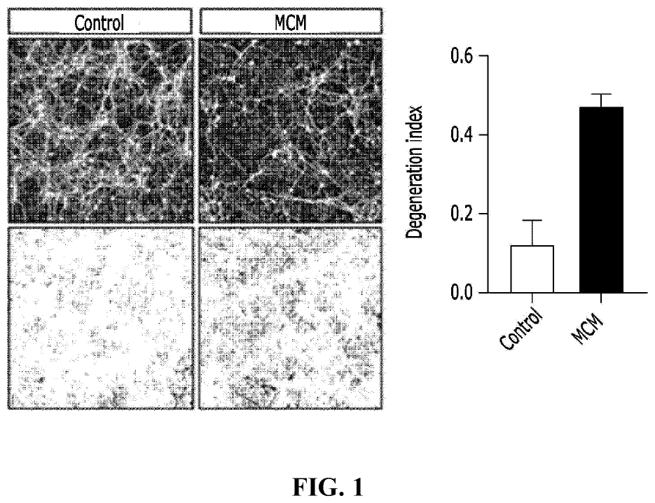

FIG. 1 shows results confirming neuronal cell death caused by neurotoxicity when a macrophage culture medium according to one experimental example of the present disclosure is treated to neurons.

Referring to FIG. 1, it was observed that neurotoxicity increased upon treatment with the macrophage culture medium.

### Experimental Example 2

In a study involving the inventors of the present disclosure, it was reported that, when a culture medium obtained after treating macrophages co-cultured with dorsal root ganglion sensory neurons with cyclic AMP (cAMP) was treated to neurons, the culture medium enhanced the regenerative ability of neurons. To confirm the degree of neurotoxicity of macrophages having regenerative ability, dorsal root ganglion sensory neurons were first cultured and then cultured for 24 hours with macrophages differentiated for 4 hours. Thereafter, the medium of the cells was replaced, and the cells were cultured again for 72 hours. The culture medium was then treated to cortical cells, and after 48 hours, images of neurons were obtained through antibody staining in the same manner as in FIG. 1, and the degree of neurotoxicity was quantified.

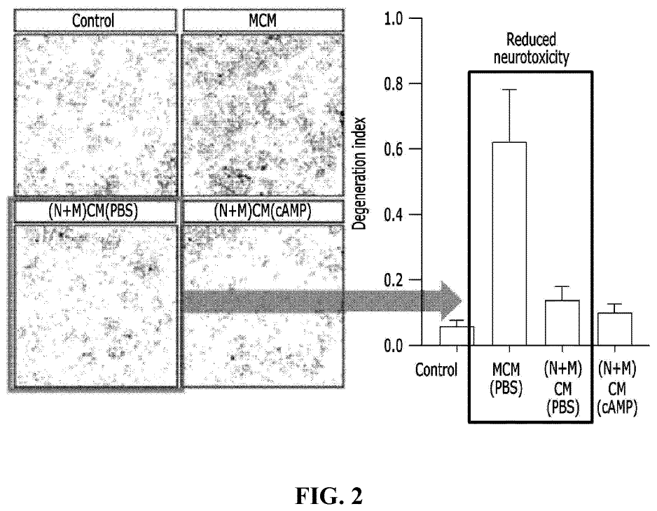

FIG. 2 shows results confirming reduced neurotoxicity when a culture medium of macrophages co-cultured with neurons according to one experimental example of the present disclosure is treated to neurons.

Referring to FIG. 2, it was observed that the culture medium of macrophages co-cultured with neurons clearly reduced neurotoxicity compared to treatment with the culture medium of macrophages cultured alone.

### Experimental Example 3

In order to confirm differences in gene expression of macrophages under conditions of macrophages cultured alone or co-cultured with neurons, RNA-sequencing capable of whole-cell gene analysis was performed. The macrophage culture conditions were the same as those in Experimental Examples 1 and 2.

The cultured macrophages were treated with Qiazol (Qiagen, Dusseldorf, Germany) to lyse the cells, mixed with chloroform in an amount of 20% of the Qiazol volume, and then centrifuged. After the supernatant was separated, 1 ml of isopropyl alcohol and yeast tRNA were treated, followed by incubation at a low temperature for 1 hour. After incubation, the mixture was centrifuged for 30 minutes, and the precipitated RNA was washed with a 70% ethanol solution and then dried at room temperature for 5 minutes. The dried RNA was diluted in RNase-free water, and RNA-sequencing was performed. FASTQ files obtained after RNA-sequencing were used to isolate differentially expressed genes (DEGs) using the DESeq2 program of the R program. The differentially expressed genes were analyzed using IPA (Ingenuity Pathway Analysis) and GSEA (Gene Set Enrichment Assay) to analyze genes increased in macrophages co-cultured with neurons and signaling mechanisms related thereto.

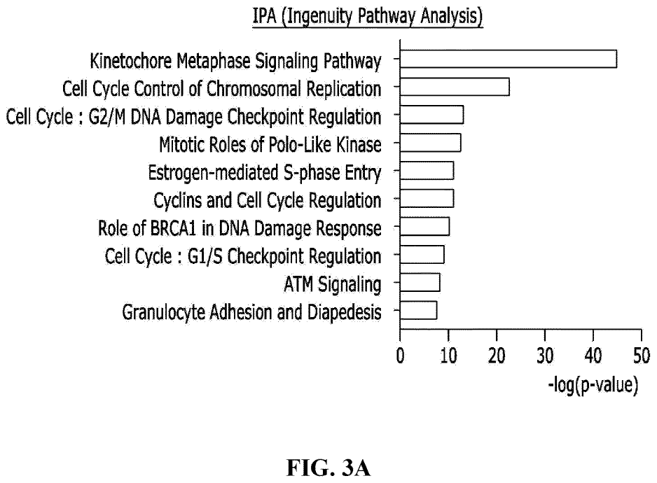

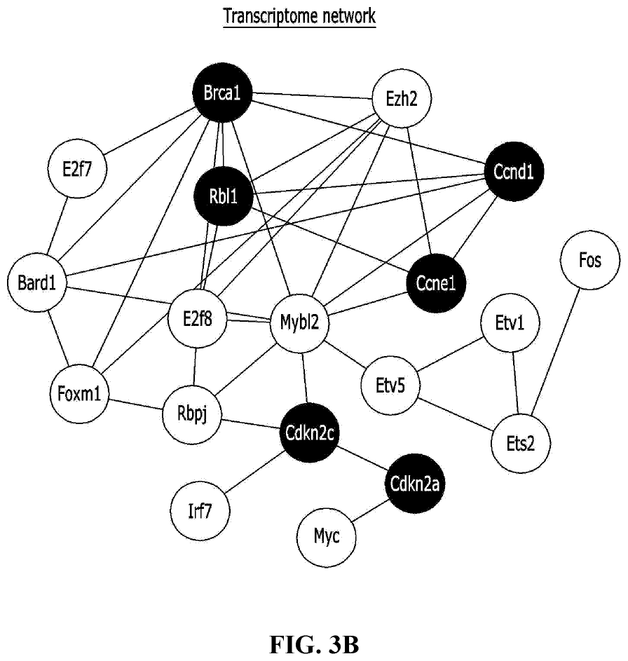

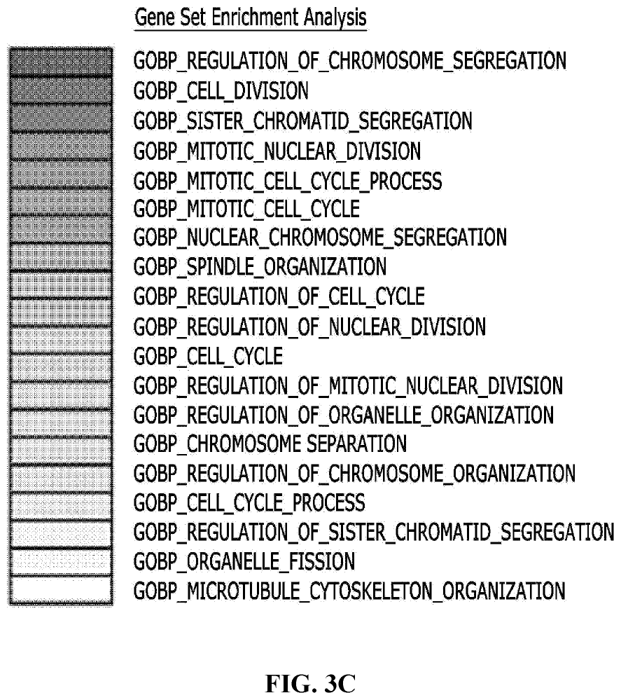

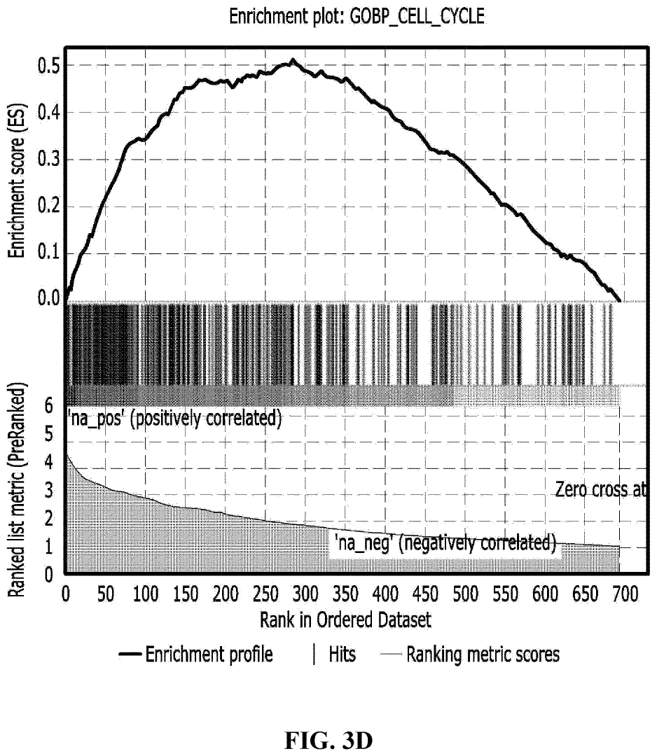

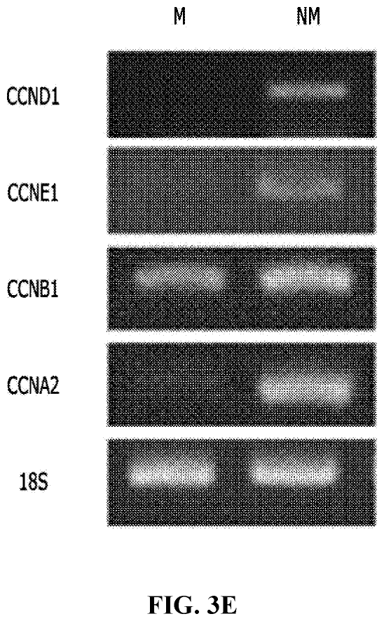

FIG. 3A shows results confirming an increase in cell cycle-related mechanisms by analyzing genomes increased in co-cultured macrophages using IPA (Ingenuity Pathway Analysis).

FIG. 3B shows results confirming, by examining a network of genomes increased in co-cultured macrophages using Cytoscape, that cell cycle-related genes are included in core proteins of the network.

FIG. 3C shows results confirming an increase in cell cycle-related mechanisms by analyzing genomes increased in co-cultured macrophages using a GSEA database.

FIG. 3D shows results confirming that many genes are associated with cell cycle mechanisms in GSEA database analysis.

FIG. 3E shows results confirming, through PCR, that cell cycle genes are increased in co-cultured macrophages in transcriptome analysis.

Referring to FIGS. 3A to 3E, as a result of the analysis, it was confirmed that cell cycle-related genes and mechanisms were increased in macrophages co-cultured with neurons.

### Experimental Example 4

Among cell cycle genes that regulate neurotoxicity, it has been reported that expression of an AURK (Aurora kinase) gene is inhibited by a cell cycle inhibitor, and that inhibition of the AURK gene changes the phenotype of macrophages. Accordingly, RNA-seq analysis was performed based on Experimental Example 3.

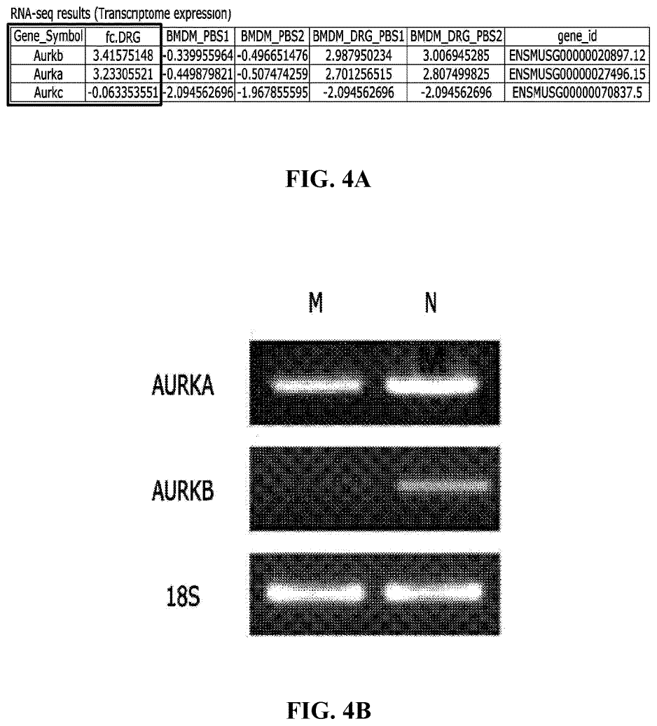

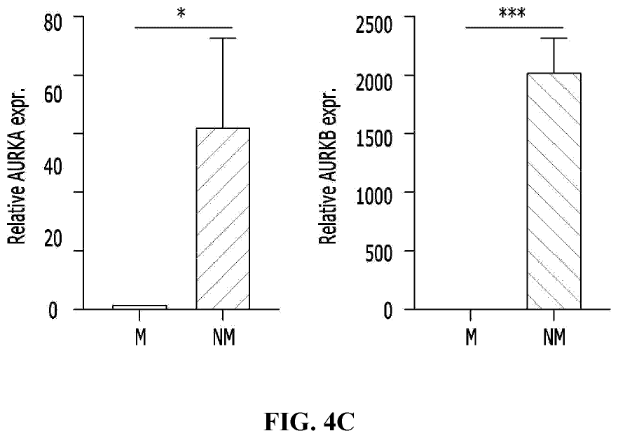

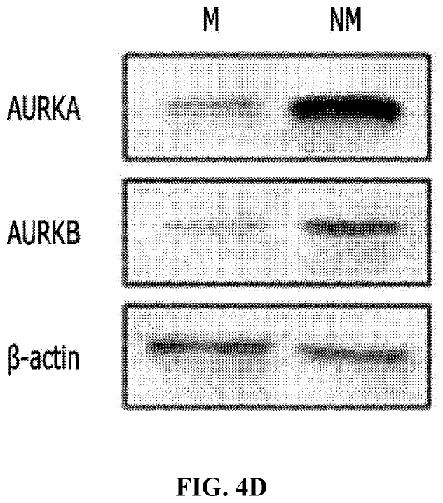

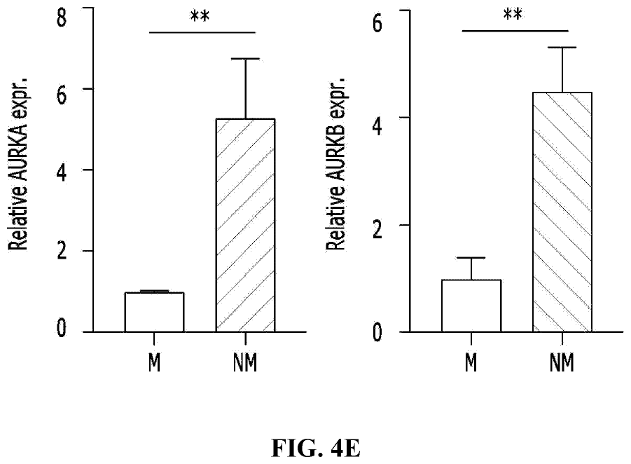

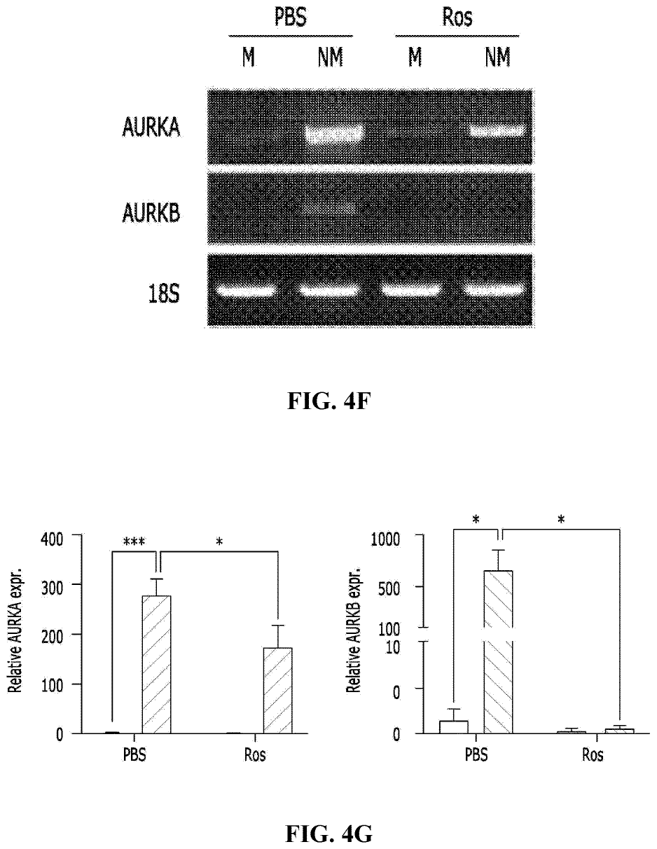

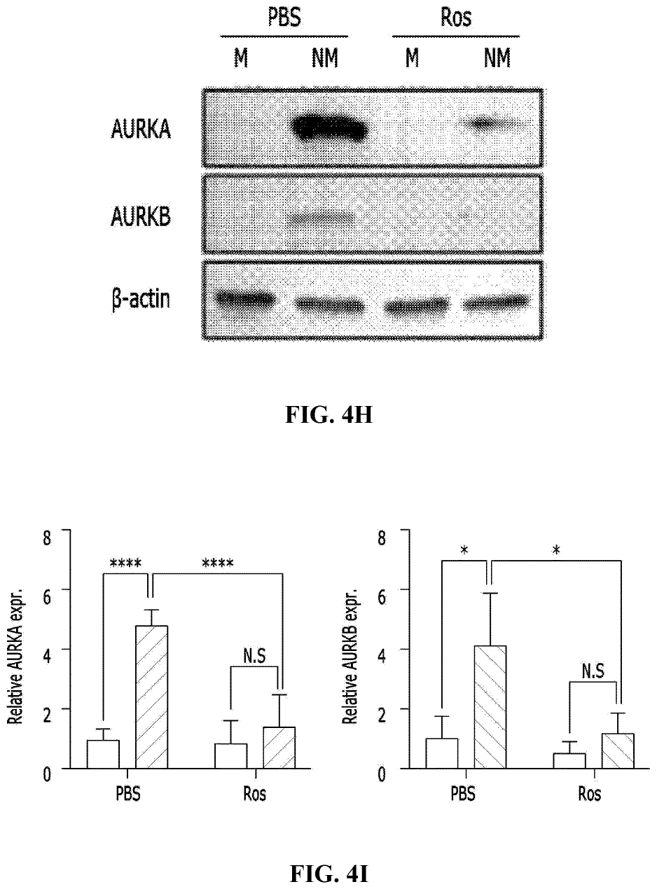

FIG. 4A shows results of observing expression of AURKA, AURKB, and AURKC in transcriptome results.

FIG. 4B shows results of observing, through PCR, that expression of AURKA and AURKB is increased in co-cultured macrophages.

FIG. 4C shows the quantified real-time PCR results corresponding to FIG. 4b, confirming increased expression of AURKA and AURKB in co-cultured macrophages.

FIG. 4D shows results of observing, through Western blot, that expression of AURKA and AURKB is increased in co-cultured macrophages.

FIG. 4E shows results obtained by quantifying the Western blot results of FIG. 4D using ImageJ, thereby confirming that expression of AURKA and AURKB is increased in co-cultured macrophages.

FIG. 4F shows results of observing, through PCR, that expression of AURKA and AURKB, which had been increased in co-cultured macrophages, is decreased upon treatment with a cell cycle inhibitor.

FIG. 4G shows the quantified real-time PCR results corresponding to FIG. 4f, confirming increased expression of AURKA and AURKB in co-cultured macrophages.

FIG. 4H shows results of observing, through Western blot, that expression of AURKA and AURKB, which had been increased in co-cultured macrophages, is decreased upon treatment with a cell cycle inhibitor.

FIG. 4I shows results obtained by quantifying the Western blot results of FIG. 4H using ImageJ.

Referring to FIGS. 4A to 4I, through PCR (polymerase chain reaction), which can confirm gene expression of cells, and Western blot, which can confirm protein expression, it was confirmed that expression of AURKA (Aurora kinase A) and AURKB (Aurora kinase B) among the AURK family was increased in macrophages when neurons and macrophages were co-cultured. In addition, it was observed that expression of AURKA and AURKB decreased when a cell cycle inhibitor was treated.

### Experimental Example 5

In order to confirm whether macrophage neurotoxicity changes when the functions of the cell cycle regulatory genes AURKA and AURKB are inhibited, changes in neurotoxicity were confirmed after treatment with inhibitors specific to AURKA and AURKB, respectively.

After neurons and macrophages were co-cultured, the inhibitors were treated, followed by culturing for 24 hours. Thereafter, the medium was replaced with a fresh medium, and the cells were cultured for 72 hours. The culture medium obtained after culture was treated to cortical neurons, and the degree of neuronal damage was quantified. As a result, it was confirmed that macrophage neurotoxicity increased when the AURK inhibitor was treated. Conversely, in order to observe whether neurotoxicity changes when expression of AURK is increased, a plasmid including an AURK gene was introduced into macrophages, and changes in neurotoxicity were observed. First, macrophages were transfected with plasmids encoding AURKA or AURKB using electroporation. After electroporation, the cells were incubated in culture medium, and the medium was replaced with fresh medium after 4 hours. The macrophages were then further cultured for 72 hours.

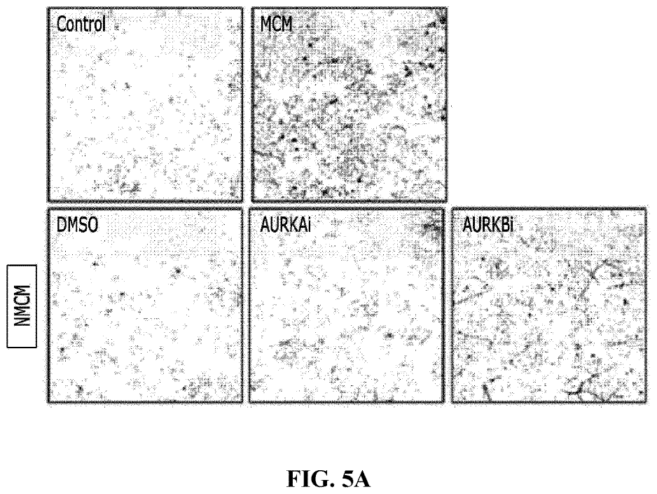

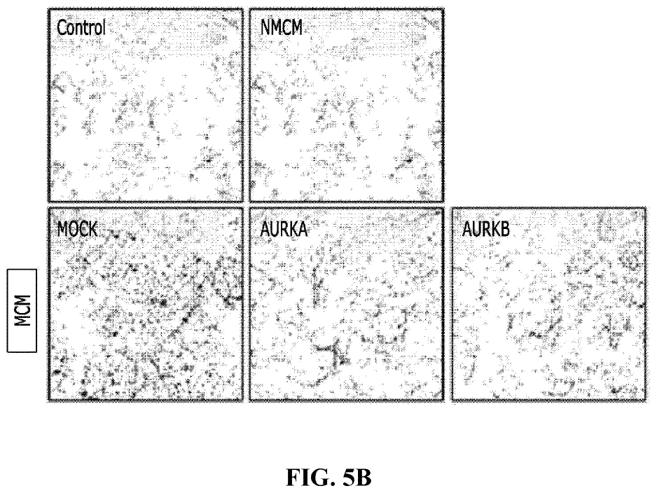

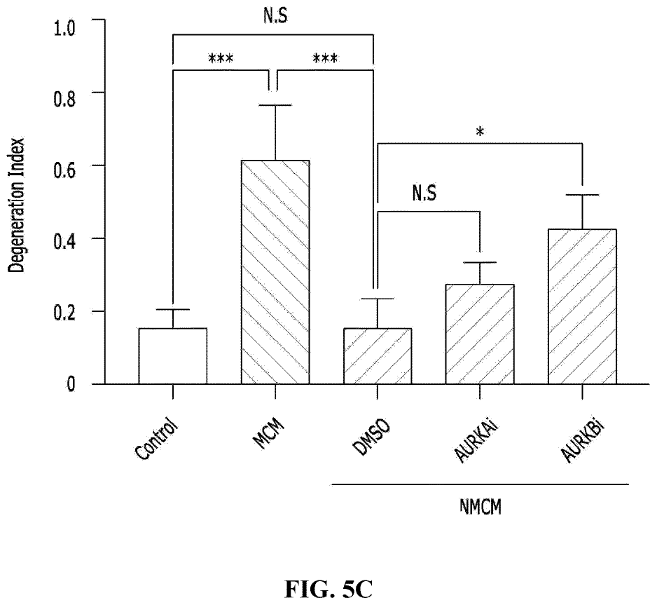

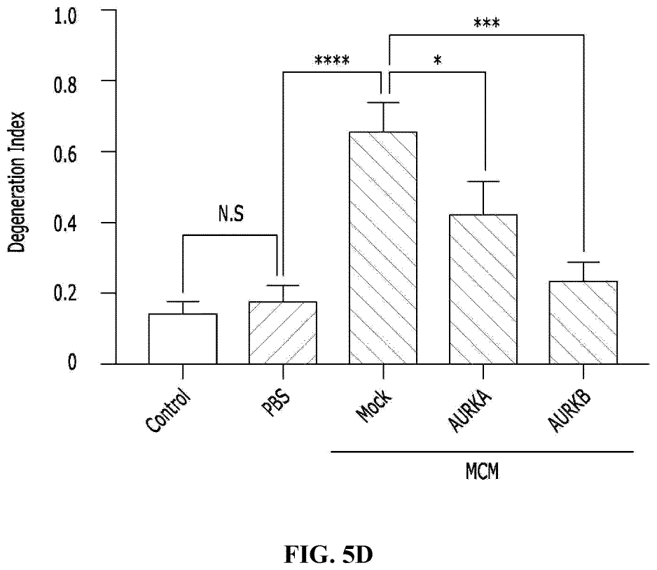

FIG. 5A shows results confirming that neurotoxicity is increased when expression of AURKA or AURKB is inhibited in macrophages using specific inhibitor, a culture medium is obtained, and the culture medium is treated to neurons.

FIG. 5B shows results confirming that neurotoxicity is decreased when a plasmid including an AURKA or AURKB sequence is introduced into macrophages not co-cultured with neurons (macrophages cultured alone) to overexpress AURKA or AURKB, respectively, a culture medium is obtained, and the culture medium is treated to neurons.

FIG. 5C shows results obtained by quantifying the image obtained in FIG. 5A and then calculating a D.I. (Degeneration Index).

FIG. 5D shows results obtained by quantifying the image obtained in FIG. 5B and then calculating a D.I. (Degeneration Index).

Referring to FIGS. 5A to 5D, as a result of treating cortical neurons with the culture medium of cultured macrophages and quantifying the degree of neuronal damage, it was observed that neurotoxicity was decreased when expression of AURKA or AURKB was increased, compared to macrophages in which gene expression was not increased.

### Experimental Example 6

After inducing a photothrombotic stroke model, tissue immunostaining was performed on day 7, when degeneration caused by macrophages increases, to confirm the presence of damage. In order to confirm expression of AURKA and AURKB after stroke induction, tissues were obtained, which were collected from mice in which photothrombotic stroke was not induced and mice in which photothrombotic stroke was induced, respectively. In particular, in the induced mice, tissues were obtained from the injured region (Ipsi) and the contralateral region (Contra).

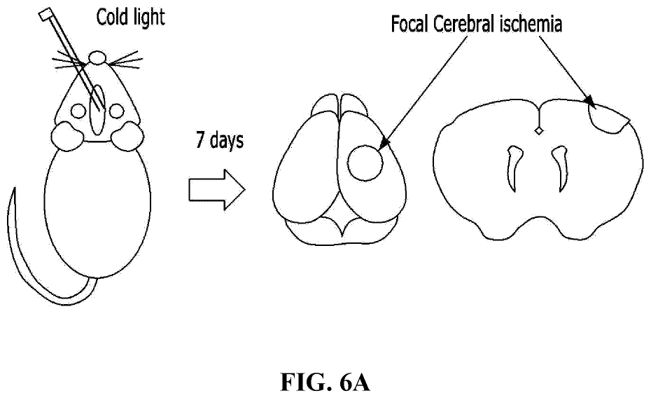

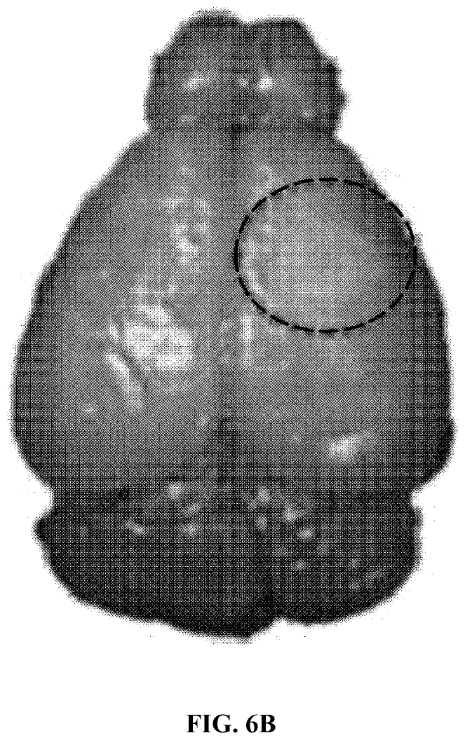

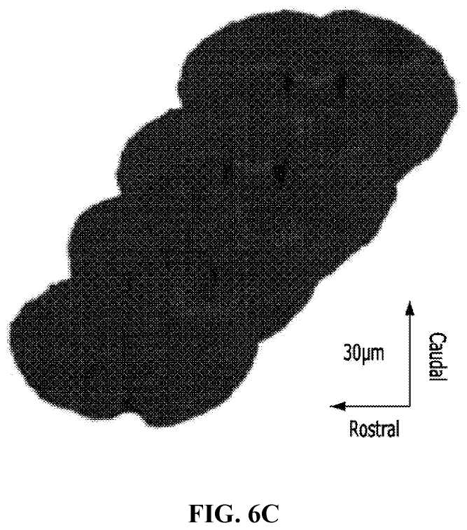

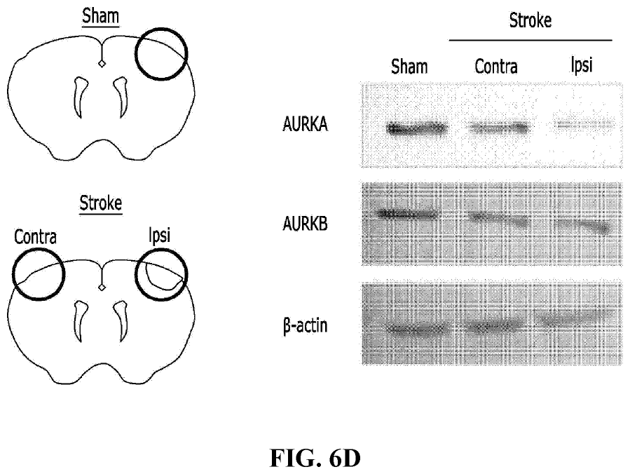

FIG. 6A is a schematic illustration of a process of inducing photothrombotic stroke in mice, and includes the meaning that damage can be observed 7 days after inducing stroke in mice.

FIG. 6B shows results confirming that damage occurred, as indicated by a circle, as a result of observing the brains of mice after 7 days.

FIG. 6C shows results confirming that a lesion occurred through CV (Cresyl Violet) staining after sectioning tissue.

FIG. 6D shows results of observing decreased expression of AURKA and AURKB in an injured region by performing Western blot using tissues obtained from Sham, Stroke Contra, and Stroke ipsi regions 7 days after injury.

Referring to FIGS. 6A to 6D, as a result of collecting tissues and analyzing the amount of protein, it was confirmed that expression of AURKA and AURKB was decreased in the injured region.

The aforementioned description of the present disclosure is to be exemplified, and it will be understood by those skilled in the art that the present disclosure may be easily modified in other detailed forms without changing the technical spirit or required features of the present disclosure. Therefore, it should be appreciated that the examples described above are illustrative in all aspects and are not restricted. For example, each component described as a singular form may be implemented in a distributed manner, and components described as being distributed may also be implemented in a combined form.

The scope of the present disclosure is represented by appended claims to be described below rather than the detailed description, and it is to be interpreted that the meaning and scope of the claims and all the changes or modified forms derived from the equivalents thereof come within the scope of the present disclosure.

1. A method for inhibiting macrophage neurotoxicity, comprising increasing expression of Aurora kinase in macrophages.

2. The method for inhibiting macrophage neurotoxicity of claim 1,
wherein the Aurora kinase comprises one selected from the group consisting of AURKA, AURKB, and combinations thereof.

3. The method for inhibiting macrophage neurotoxicity of claim 1,
wherein the increasing of the expression comprises co-culturing the macrophages with neurons.

4. The method for inhibiting macrophage neurotoxicity of claim 3,
wherein the neurons comprise one selected from the group consisting of dorsal root ganglion sensory neurons, hippocampal neurons, dopaminergic neurons, motor neurons, cerebellar granule cells, thalamic neurons, spinal cord neurons, and combinations thereof.

5. A biomarker for diagnosing a neurodegenerative disease, comprising Aurora kinase as an active ingredient.

6. The biomarker for diagnosing a neurodegenerative disease of claim 5,
wherein the Aurora kinase comprises one selected from the group consisting of AURKA, AURKB, and combinations thereof.

7. The biomarker for diagnosing a neurodegenerative disease of claim 5,
wherein, when downregulation of an expression level of the Aurora kinase is observed, a subject is determined to have a neurodegenerative disease.

8. The biomarker for diagnosing a neurodegenerative disease of claim 5,
wherein the neurodegenerative disease comprises one selected from the group consisting of stroke, Alzheimer's disease, Parkinson's disease, Huntington's disease, and combinations thereof.

9. The biomarker for diagnosing a neurodegenerative disease of claim 5,
wherein the neurodegenerative disease comprises stroke.

10. A method for screening an inhibitor of macrophage neurotoxicity, comprising:
treating macrophages with a candidate substance;
measuring an expression level of Aurora kinase in the macrophages; and
selecting the candidate substance as an inhibitor of macrophage neurotoxicity when expression of the Aurora kinase is increased.

11. The method for screening an inhibitor of macrophage neurotoxicity of claim 10,
wherein the Aurora kinase comprises one selected from the group consisting of AURKA, AURKB, and combinations thereof.
